(19) United States
(12) Patent Application Publication
(10) Pub. No.: US 2021/0137601 A1
(43) Pub. Date: May 13, 2021

(54) DEVICE AND METHOD FOR IMAGING DURING IMPLANTATION OF RETINA IMPLANTS

(71) Applicant: Carl Zeiss AG, Oberkochen (DE)

(72) Inventors: Johannes KINDT, Weimar (DE); Rudolf Murai VON BUENAU, Jena (DE); Tobias SCHMITT-MANDERBACH, Kempten (DE)

(73) Assignee: Carl Zeiss AG, Oberkochen (DE)

(21) Appl. No.: 17/053,180

(22) PCT Filed: May 6, 2019
(86) PCT No.: PCT/EP2019/061497

§ 371 Date: Nov. 5, 2020

(30) Foreign Application Priority Data
May 6, 2018 (DE) 10 2018 110 842.7
Aug. 17, 2018 (DE) 10 2018 213 872.9

(51) Int. Cl. A61B 34/10; A61B 3/10; A61B 3/00; A61B 3/13; A61B 90/20; A61B 90/00

(52) U.S. Cl. CPC A61B 34/10; A61B 3/102; A61B 3/0025; A61B 3/0058; A61B 3/13; A61B 2090/3735; A61B 90/37; A61B 2034/105; A61B 2034/107; A61B 2034/104; A61B 90/20

(57) ABSTRACT

Methods and devices for visualising an implant in a retina are provided. A 2D image of the retina is taken and OCT scans of the retina and implant are carried out. Based thereon, the implant and retina are visualised.

The present application relates to apparatuses and methods for imaging within the scope of implanting retinal implants, which apparatuses and methods can serve, in particular, to prepare such an implantation or to provide assistance during the implantation. In this context, it should be noted that the implantation itself, i.e., the surgical procedure, is not part of the subject matter of the present application. In particular, the presented apparatuses and methods are non-invasive, i.e., imaging is implemented from the outside, in particular by means of electromagnetic waves such as light that passes through the pupil of an eye.

Retinal implants are apparatuses which are implanted in the retina of the patient's eye or which are fastened to the retina in order to fulfill a specific therapeutic or prosthetic function for the relief of ocular diseases. By way of example, implants can administer medicaments, can exert a mechanical function such as stabilization or fastening or else can output electrical stimulation in response to incident light in order to at least partly replace the function of light-sensitive cells (rods, cones), which normally work in the retina in order to convert light into nerve impulses.

When implanting such retinal implants, accurate positioning of the retinal implant in or on the retina is required so that the implant can fulfill the desired function and so that damage, for example to healthy parts of the retina or other parts of the eye, is avoided.

Surgical microscopes are frequently used to assist a surgeon with the implantation of retinal implants. These show an image of the interior of the eye even during the operation, said image being captured through the pupil of the eye to be operated on. Substantially only a two-dimensional display is obtained in this case even if stereo microscopes are used, since the constraint that the light rays must pass through the pupil of the eye leads to a stereo basis that is very small at best. In particular, the height of the implant above the retina cannot be identified or measured or can only be identified or measured poorly in this case.

An example of such a surgical microscope is the OPMI Lumera® 700 by Zeiss.

Modern surgical microscopes combine optical image recording with optical coherence tomography (OCT).

Optical coherence tomography is an optical imaging method which provides depth information for semi-transparent objects. Line scans, in particular, are recorded in this case; these yield depth profiles along the scan line. However, depth profiles are then conventionally only displayed along a line in this case, making it difficult for a surgeon to identify a positional relationship in all spatial directions, i.e., a three-dimensional positional relationship, between the implant and the retina.

Here, optical coherence tomography is used to identify anatomical structures such as the various retinal layers and pathological structures such as lesions and to identify surgical instruments such as cannulas or tweezers in the case of intra-operative OCT. For comparatively extensive retinal implants, which moreover are usually non-transparent and partly cover the retina, such techniques only have limited use.

It is therefore an object to provide improved apparatuses and methods for imaging within the scope of implanting retinal implants.

This object is achieved by a method as claimed in claim 1 and an apparatus as claimed in claim 17. The dependent claims further define embodiments.

According to a first aspect of the invention, a method for visualizing an implantation of a retinal implant is provided, comprising:

recording a 2D image of a retina and of an implant,

carrying out an OCT scan, i.e., a scan by means of optical coherence tomography, of the retina and an OCT scan of the implant, and

visualizing the implant and the retina on a display on the basis of the 2D image and the OCT scan.

In this way, a surgeon can be assisted during and, where necessary, also prior to the implantation.

It should be observed that, as already mentioned at the outset, the operation itself is not part of the claimed method and the method is carried out non-invasively by way of recordings through the pupil of the eye.

It should be noted that the recording of the 2D image and the OCT scan of the implant in exemplary embodiments serves, in particular, to determine the position of the implant relative to the retina and/or to determine a tilt of the implant. Therefore, the phrase “OCT scan of the implant” should not be understood to mean that the entire implant needs to be scanned. Rather, a single scan line over the implant is sufficient in many cases to determine the height of the implant above the retina and/or the tilt of the implant. Nor does the phrase “OCT scan of the retina” mean that the entire retina is scanned. In many cases, it may be sufficient for only a part of the retina or, likewise, only a single scan line to be scanned. Here, it is also possible to resort to earlier OCT scans of the retina. The 2D image can be recorded, in particular, during the operation by means of a surgical microscope.

The visualization of the implant can comprise a display of an avatar of the implant.

By using an avatar for visualizing the implant, the latter can be represented in accordance with the real shape of the implant, simplifying an identification of the positional relationship between implant and retina. Here, parts of the implant could be masked or highlighted, for example, or only the outlines of the implant could be displayed. Here, the real shape of the implant is known—e.g., from the manufacturer data—and therefore need not be ascertained separately as a rule even if, as a matter of principle, this is possible where necessary by means of image recordings and/or OCT scans in some exemplary embodiments.

Here, an avatar should be understood to be a graphical representation of the implant which, in terms of its shape, corresponds to the shape of the implant or, in the case of a multi-part implant, a part thereof. During the operation, the avatar is displayed in respect of position and alignment in accordance with the real position and alignment of the implant, in the eye, within the measurement accuracy.

The display of the avatar can comprise a display of an avatar of a structural component of the implant and an optional display of an avatar of a functional component of the implant.

This allows a visualization of the relative position of a functional component of the implant, too, even if only the structural component of the implant is currently actually implanted in the eye. Here, a structural component of an implant should be understood to be a part of an implant which fulfills structural functions and, in particular, serves to hold, e.g., fasten, the implant at a desired position on or in the retina. The functional component fulfills the actual function of the implant, for example the generation of electrical pulses in response to incident light or the administration of medicaments to the retina.

In some implants, the implant can also have a first configuration and a second configuration. The implant is in the first configuration for the implantation procedure and subsequently brought into the second configuration post implantation. By way of example, the second configuration can be an unfolded or expanded configuration, which is adopted by the activation of springs or other elastic elements.

In some embodiments, a choice can be made for the avatar between a visualization of the first configuration and a visualization of the second configuration. Thus, the implant can be visualized in the second configuration, adopted following the implantation, even though it actually still is in the first configuration; this can simplify positioning.

The method can further comprise determining a relative position of the implant in the 2D image of the retina and determining a scan line of the OCT scan of the retina and a scan line of the OCT scan of the implant on the basis of what was identified.

By carrying out two such OCT scans with the scan lines by means of optical coherence tomography, it is possible to accurately ascertain a distance between the implant and the retina.

Accordingly, the method can further comprise determining a distance between the implant and the retina. Then, the method can further comprise a display of the distance on the display. Here, the distance can be displayed directly as a numerical value, for example. However, a display by means of a false color representation is also possible. By way of example, the aforementioned avatar of the implant can be colored green in the case of a large distance, can be colored yellow in the case of a shorter distance and can be colored red in the case of a distance at or near zero. Displaying the distance is therefore not restricted to a certain type of display. Thus, the described method also facilitates quantitative measurements of the positional relationship between implant and retina.

The visualization of the retina can comprise a visualization of a part of the retina located below the implant on the basis of a previous OCT scan.

By using a previous OCT scan of the retina it is possible to visualize both retina and implant, even if a part of the retina located under the implant is currently not visible for the image recordings.

The visualization can comprise a visualization of regions of the retina suitable for implantation. This simplifies the selection of a suitable site for the implantation.

The visualization can comprise a visualization of a penetration of fastening means of the implant into the retina.

Such a visualization of fastening means allows better positioning of the retinal implant, in particular in respect of the positioning in a direction perpendicular to a local plane of the retinal surface. Here, a local plane is a plane that locally approximates the (generally curved) retinal surface. In particular, it can be a tangential plane at a point of the retina.

The visualization can further comprise an output of an indication as to whether the penetration depth of the fastening means is correct. This simplifies correct fastening of the implant.

The visualization can also comprise a simulation of a mechanical reaction of the retina to the implant and a visualization of the simulated mechanical reaction.

Prior to the implantation, the method can further comprise: carrying out a virtual operation procedure with a further visualization for establishing a planned implant position. In this case, the visualization comprises a display of the planned implant position. This assists the implantation at the planned implant position.

The further visualization within the scope of the virtual operation can be carried out on the basis of a user input for controlling the implant, a 2D image of the retina, and an OCT scan of the retina.

The method can further comprise:

prior to the implantation, creating annotations, wherein the visualization comprises a display of the annotations. Here, annotations are inputs of a user, e.g., a surgeon, which are made for certain parts of image recordings, OCT scans or the like and which can then be visualized at the correct position.

The method can further comprise augmenting the visualization on the basis of the data obtained prior to the implantation. The data obtained prior to the implantation can comprise a recording of the fundus and/or data from retinal angiography. Thus, a displayed image region can be enlarged using data from the fundus recording or additional information, for example from retinal angiography, can be displayed. This can be done optionally.

According to a second aspect of the invention, an apparatus for visualizing an implantation of a retinal implant is provided, comprising:

a surgical microscope with a camera for recording a 2D image of a retina and of an implant, an OCT device, and

a computing device, wherein the computing device is configured to drive the OCT device to carry out an OCT scan of the retina and an OCT scan of an implant and to drive a display to visualize the implant and the retina.

The apparatus can be configured to carry out one or more of the above-described methods, in particular by an appropriate design, e.g., programming, of the computing device.

The invention is explained in greater detail below on the basis of preferred exemplary embodiments with reference to the accompanying drawings. In detail:

FIG. 1 shows a block diagram of an apparatus in accordance with one exemplary embodiment,

FIG. 2 shows a flowchart for elucidating a method in accordance with one exemplary embodiment,

FIG. 3 shows a schematic view of an eye during an implantation for elucidating exemplary embodiments,

FIG. 4 shows an example of a visualization,

FIG. 5 shows an example of an implant with two parts, as is used in some exemplary embodiments,

FIG. 6 shows a perspective view of an eye during an operation for elucidating exemplary embodiments,

FIG. 7 shows a visualization in accordance with a further exemplary embodiment,

FIG. 8 shows a visualization in accordance with a further exemplary embodiment,

FIG. 9 shows an elucidation of various techniques in accordance with various exemplary embodiments during an operation,

FIG. 10 shows an elucidation of various techniques in accordance with some exemplary embodiments for planning an operation, and

FIG. 11 shows an elucidation of various techniques in some exemplary embodiments during an operation, which have been preceded by planning as in FIG. 10.

Various exemplary embodiments are explained in detail below. These are only illustrative and should not be construed as limiting.

Variations, modifications, and details that have been described for one of the exemplary embodiments are also applicable to other exemplary embodiments, unless indicated otherwise, and are therefore not described repeatedly. Features of various exemplary embodiments can also be combined with one another. Thus, various techniques for providing an improved visualization during an eye implantation are described below; these are applicable individually or in combination with one another.

FIG. 1 shows an apparatus 10 for imaging within the scope of an implantation of retinal implants in accordance with one exemplary embodiment. The apparatus 10 comprises a microscope 12 with a camera for the provision of image recordings of an eye, in particular 2D images, i.e., images without depth information. The microscope 12 can also be a stereo microscope. However, as mentioned at the outset, the image is recorded through the pupil of an eye, and so the stereo basis of the recording is so small that substantially a two-dimensional image is also produced in this case, at best with little depth information. Moreover, the apparatus 10 comprises an OCT device 11 for optical coherence tomography (OCT). The OCT device 11 can be integrated in the microscope 12 in conventional fashion, for example like in the Zeiss microscope mentioned at the outset.

The apparatus 10 further comprises a computing device 13, which controls the OCT device 11 and the microscope 12, for example the camera of the microscope 12, and which receives image information from the camera of the microscope 12 and from the OCT device 11. The computing device 13 creates a visualization of the eye on the basis of this information, wherein an avatar is used to visualize an implant which should be implanted within the scope of an operation or which is currently being implanted. The visualization is then displayed on a display 15. Here, the display can be integrated in the microscope 12 such that a user, such as a surgeon, sees the visualization when looking into the microscope. A separate display is possible in addition or as an alternative thereto. Various aspects of the visualization will be explained in more detail below. The computing device 13 can be a computer which comprises one or more appropriately programmed processors. In addition or as an alternative thereto, it can be realized by means other suitable components, such as application-specific integrated circuits (ASICs), field-programmable gate arrays (FPGAs), digital signal processors, and the like.

In the exemplary embodiment of FIG. 1, the OCT device 11 can be used to capture, in particular, a structure of the retina of the eye, into which the implant should be inserted. This can already be done in the run-up to the operation for planning purposes. Moreover, since OCT data supply depth information, a current distance of the implant from the retina and also a tilt of the implant can be captured by means of the OCT device 11. To this end, an OCT line scan can be carried out next to the implant and an OCT line scan can be carried out over the implant, for example during the operation, as will likewise be explained in more detail below. Here, the position of the implant can be determined by means of image processing on the basis of images supplied by the camera of the microscope 12.

Then, an avatar of the implant can be always displayed at the detected position during the operation. Moreover, a tilt of the implant can be measured continuously by means of the line scan over the implant; this is likewise displayable in real time.

FIG. 2 shows a flowchart of an exemplary embodiment of a corresponding method. By way of example, the method can be carried out using the apparatus 10 of FIG. 1 and the explanations given there apply accordingly to the method.

In step 20 of FIG. 2, a 2D image of the retina of the eye, possibly with an implant located thereabove, is recorded. The explanations made for the camera of the microscope 12 also apply here; i.e., the image need not be a pure 2D image but may also have been recorded by a stereo camera, for example. In step 21, an OCT scan of the retina and, where applicable, the implant is recorded, as explained for the OCT device 11 of FIG. 1. In step 22, the implant is visualized and displayed together with the retina, as explained for the computing device 13 and the display 15 of FIG. 1.

Examples of such visualizations are now explained in more detail.

To this end, FIG. 3 shows a schematic view of an eye when inserting an implant 36. Here, FIG. 3 shows a view of the eye as may have been recorded by the camera of the microscope 12 of FIG. 1 or in step 20 of FIG. 3 as a 2D image.

FIG. 3 features the eye with sclera 32, iris 31, and the retina 35 visible through the pupil. A surgical instrument 30 is introduced into the eye by way of a trocar 37 in order to position an implant 36.

The implant 36 is identified in the 2D image corresponding to FIG. 3 by way of image processing algorithms. OCT scans are carried out on the basis of the relative position of the implant thus identified. By way of example, a first OCT scan is carried out along a line 33 over the implant 36 and a second OCT scan 34 is carried out adjacent to the implant 36 over the retina 35. In this way, it is possible to determine the tilt of the implant and the position of the implant 36 relative to the retina 35 in a direction perpendicular to the retina 35. Below, this direction perpendicular to the retina is also referred to as z-direction while the image plane of the image of FIG. 3, which approximately corresponds to the plane of the retina 35 (if a planar retina is assumed), is referred to as xy-plane.

FIG. 4 shows an example of a visualization, as is able to be created on the basis of the image recording and the OCT scan of FIG. 3. Here, an avatar 41 of the implant is displayed above a representation 40 of the retina in a perspective view. Here, the representation 40 of the retina is partly displayed as an OCT slice image. From this, it is possible to identify the structure of the retina, for example a point of sharpest vision, and the implant can be positioned accordingly. Here, the position of the avatar 41 is continuously updated to the actual position of the implant 36 during the operation. By way of example, if the implant 36 is moved laterally over the retina 35, i.e., in the xy-direction according to the definition above, the avatar 41 moves accordingly. Moreover, the representation 40 of the retina is always displayed adjacent to the implant. If the implant is moved toward the retina or away from the retina then this is captured by the OCT scans along the lines 33, 34 and the position of the avatar 41 is continuously updated accordingly in the process. Moreover, annotations can also be displayed, for the purposes of which an arrow 42 is displayed as an example. In some exemplary embodiments, such annotations can be created freely in advance by a user, for example in order to mark certain regions of the retina. They can then optionally be displayed in the visualization. This will later be explained in more detail with reference to FIG. 10.

Since retinal implants are typically not transparent, the region of the retina directly under the implant cannot be captured at the same time as the implant by means of optical coherence tomography. In this case, only the retinal structure adjacent to the implant is displayed, which retinal structure can be captured by OCT scans such as the scan along the line 34, or information from previous OCT scans when the implant 41 was at a different position is used to visualize the retina in full.

Some implants consist of two or more parts. As an example, FIG. 5 illustrates an implant which comprises a structural component 50 and a functional component 51. The structural component 50 serves to fasten the implant in or on the retina. The functional component 51 serves to provide the actual function of the implant, for example to administer medicaments, stimulate nerves or the like. The functional component 51 is held by the structural component 50.

When such an implant is implanted, the structural component 50 is initially fastened in or on the retina and then the functional component 51 is inserted into the structural component 50. The insertion of the structural component 50 into the eye by means of an aforementioned surgical instrument 30 through the trocar 37 is schematically illustrated in FIG. 6. Similar to FIG. 3, FIG. 6 simultaneously shows an example of a 2D image, as is recordable by the camera of the microscope 12.

As explained with reference to FIGS. 3 and 4, a visualization can also be created in this case by combining OCT scans and image recording. Additionally, an avatar of the functional component 51 can be displayed in this visualization. An example of such a visualization is illustrated in FIG. 7.

Similar to FIG. 4, FIG. 7 shows a visualization in which an avatar of an implant is displayed above a representation 40 of the retina. In this case, the avatar of the implant consists of two parts, specifically an avatar 70 of the structural component and an avatar 71 of the functional component. In this case, the avatar 71 of the functional component is able to be shown and hidden so that, optionally, the actual situation during the current implantation of the structural component or, additionally by way of the avatar 71, the later position of the functional component can be displayed. This can simplify positioning of the implant. Since, as explained, the functional component in fact fulfills the actual function of the implant, the positioning thereof relative to the features of the retina (e.g., relative to specific parts of the retina or diseased parts of the retina), in particular, may be of importance. This positioning is made easier by the avatar 71 of the functional component since the surgeon can in this case exactly identify the subsequent position of the functional component. Apart from the fact that the avatar of the functional component 71 is additionally displayed (possibly optionally displayed), the visualization of FIG. 7 corresponds to the visualization already discussed with reference to FIG. 4.

When the implant is being implanted into the retina, it is moreover possible to visualize the interaction of the implant with the retina and the precise position of the implant. In particular, the interaction of the implant with the tissue of the retina can be visualized, for the purposes of which simulations can be used. To this end, as already illustrated in FIGS. 4 and 7, an avatar of the implant (possibly two-part implant as in FIG. 7) is displayed together with the retina. When the implant approaches the retina, a mathematical model of the biomechanical response of the retinal tissue to the approach of the implant, for example, can be used to display an accurate visualization of the interaction between the implant and the retina. To this end, it is possible, for example, to simulate an elastic deformation of the retinal tissue and/or of the implant, a penetration of the implant into the retina, a displacement of retinal tissue, and the like. Then, the structure of the retina obtained through OCT scans can be displayed in modified fashion on the basis of such a mathematical model. Here, it is possible, in particular, to also take account of an interaction of a functional component—not yet present within the operation at this time—such as the functional component 51 of FIG. 5; i.e., for example, it is possible to display how the retina is deformed by the functional component. Consequently, in FIG. 7, it is possible to visualize not only the avatar 71 of the functional component but also its interaction with the retina 40. As explained at the outset, it is also possible in the case of some implants to switch between a visualization in a first configuration, e.g., a configuration during an implantation, and a visualization in a second configuration, e.g., an unfolded configuration that is adopted after the implantation has taken place.

As mentioned, it is also possible to visualize the penetration of the implant into the retina. This is now explained with reference to FIG. 8, which shows a further example of an implant and the visualization thereof.

FIG. 8 shows a structural component 50 which in this case has fastening legs 80, which can be embodied as clips or retinal tacks or the like and by means of which the implant is anchored or held in the retina. Accordingly, the avatar 70 of the structural component is displayed together with the fastening legs in the visualization. Here, when the avatar approaches the retina 40, the position of the fastening legs 80 within the retina 40, in particular, is also displayed in the visualization. As a result of this, the correct position of the fastening legs 80, as indicated by arrows a, can be identified and, in particular, it is easier to avoid the fastening legs 80 entering structures of the retina that should not be injured. By way of example, arrow b in FIG. 8 shows part of the implant that has no fastening leg and therefore does not interact with the retina 40.

Here, additional visualization aids can be provided. By way of example, on the basis of the position of the implant and the position of the retina, which are captured by the image recording and/or OCT scans, it is possible to establish whether a desired penetration depth of the fastening legs 80 into the retina has been reached. Should this be the case, a corresponding notice can be output on a display and/or an acoustic notice or any other form of a notice can be provided in order to draw the surgeon's attention thereto. Accordingly, a different type of notification can also be provided as an alert should a desired penetration depth have already been exceeded. This is particularly helpful if, like in the example of FIG. 8, a plurality of fastening legs are present and consequently the implant penetrates the retina at a plurality of sites, since this makes it easier for the surgeon to correctly position all fastening legs in the retina.

Additionally, an indication can also be output during the visualization, said indication indicating whether a placement with a sufficient penetration depth for fastening legs such as the fastening legs 80 or other fastening means is possible in the current position of the implant above the retina (i.e., a position in the xy-plane). In this context, it should be noted that the retina is not a flat structure with uniform thickness but can have varying thicknesses and shapes, which moreover may vary from person to person. Consequently, it may be the case that an implant cannot be placed at any desired site of the retina even if the nature of the implant requires no specific positioning. Consequently, by evaluating the thickness and structure of the retina obtained from the OCT scans, the visualization can provide the surgeon with feedback as to whether correct positioning is possible at the position in the xy-plane at which the implant is currently situated. It is also possible to provide a notification about the sites of the retina at which the correct positioning can be implemented, for example with a sufficient penetration depth of fastening legs. By way of example, displays of words (such as placement OK, placement too high, too far to the left, too far to the right, too low, etc.) can count as visualizations; in addition or as an alternative thereto, use can also be made of color codes (for example in the form of a traffic light system) or arrows, which guide the surgeon to suitable positions. Use can also be made of a spatially resolved display, which, for example, is superposed on the retina 40 in the visualization. By way of example, the visualization of the retina 40 can be colored in a different color at locations at which positioning is possible than at locations where positioning is not possible, for example on account of a retina that is too thin.

This is also possible in the form of an advance simulation, in which, for the purposes of planning the operation, an avatar, for example, is moved over an OCT scan of the retina, in accordance with the visualizations discussed, in order to find a suitable placement for the implant already prior to the operation.

The aforementioned and further features of various embodiments are explained below with reference to the diagrams of FIGS. 9-11. Here, FIGS. 9-11 each show a multiplicity of various visualization options and assistance options for a surgeon before or during the operation. It should be noted that not all of these options need to be implemented. Rather, only one or a few of these options might also be realized in some exemplary embodiments. Here, the description of FIGS. 9-11 partly refers to the description above in order to avoid repetition.

Here, FIG. 9 shows an example of various visualization options during an operation, with no planning of the operation specific to the illustrated techniques having taken place in advance in this case. A combination with such advance planning is subsequently explained with reference to FIGS. 10 and 11.

The various techniques illustrated in FIG. 9 can be applied as real-time processes during the operation.

The illustration of FIG. 9 is subdivided into data capture, visualization, analysis and guidance. All of these aspects can occur continuously during an operation.

At 90, an image is captured by means of a camera of a surgical microscope, such as the camera of the microscope 12 of FIG. 1. At 91, the implant is then identified in the recorded images using conventional procedures of image analysis and image processing and the position of the implant in the xy-plane is thus determined. On the basis of this identification, an OCT scan over the implant (for example, corresponding to the line 33 of FIG. 3) is then carried out at 92 and an OCT scan of the retina adjacent to the implant (for example, by a scan along the line 34 of FIG. 3) is carried out at 94.

The OCT data of implant and retina thus obtained are then each de-warped. This de-warping will now be briefly explained:

If OCT images of the retina are recorded through the pupil, these are typically warped on account of differences between scan and display geometry and the optical properties of the eye (in particular, refraction upon passage through the pupil). In most OCT devices, use is made of a two-axis scan system with a galvanometer and freely movable mirrors for the purposes of steering the light beam used for optical coherence tomography and scanning it over the retina. When a back part of the human eye such as the retina is measured, the optical beam is scanned through a common point located at the nodal point of the eye. The nodal point is a point on the optical axis of the eye, at which the light beams which enter into the system and leave the system again at the same angle with respect to the optical axis appear to converge. Then, the light beam is guided over the (curved) posterior segment of the eye and consequently an image of a fan-shaped cross section of the eye is obtained. To display the scanned region, the depth information along individual scan lines (A-scans) are then converted into a rectangular brightness image (B-scan, brightness-modulated image), for the purposes of which the A-scans are typically stacked in parallel rather than said A-scans, i.e., the depth profile along the individual scan lines, being combined in a geometrically correct format, which offers a fan-shaped cross section matching the actual scan geometry. As a consequence, there is a discrepancy between the actual geometry and the displayed geometry.

The parameters and geometry of the employed OCT device, for example the OCT device 11 of FIG. 1, are known. If specific parameters of the respective eye such as axial eye length are now additionally measured, it is possible to use ray tracing techniques to de-warp the OCT images in order to fit these to the actual geometry of the eye. Both the measurement of the eye and this fit can be carried out using techniques known per se, just like the aforementioned ray tracing. In particular, this de-warping is helpful if, as explained with reference to FIG. 8, penetration depths are to be calculated accurately or if the geometric distance between the implant and structures of the retina is to be correctly determined and visualized. The de-warping of the OCT scans of both the retina and the implant is also helpful for the application of automatic recognition algorithms of machine vision in order thus to facilitate a more accurate localization and/or visualization.

Then, at 93, the z-coordinate of the implant, i.e., the height of the implant above the retina, is determined on the basis of the OCT scan at 92.

Then, a visualization can be implemented on the basis of the data thus obtained. Thus, for example, an avatar of the implant (for example, the avatar 41 of FIG. 4 or the avatar 70 of the structural component as illustrated in FIG. 7) is displayed at 95. Moreover, the structure of the retina, as represented by the reference sign 40 in FIGS. 4 and 7, is visualized at 97. Here, what is visualized can be selectable by the user, and so, for example, the visualization of the structure of the retina or of the avatar can optionally also be deactivated.

Moreover, at 96, an avatar of a functional component which is not yet physically present in the eye can be displayed, as explained with reference to FIG. 7. At 98, the OCT data of the retina can be supplemented, for example by virtue of a part of the retina shadowed from the OCT device employed by the implant also being visualized on the basis of previous OCT data, as already explained.

As likewise already explained briefly, different analysis and guide functions can be realized. Thus, a simulation can be carried out at 99 as to whether the implant topographically fits to the retina at the current xy-position. Corresponding thereto, advantageous and disadvantageous zones can be visualized at 912; i.e., whether or not the current xy-position of the retina is suitable for implantation purposes can be indicated to a surgeon or a different user in various ways, as explained. Then, this can be visualized accordingly at 912, as already explained above. By way of example, advantageous or disadvantageous zones of the retina can be labeled in color accordingly or a notification can be output, as likewise explained.

For analysis purposes, it is further possible to determine the penetration of the implant, for example of fastening legs or other fastening means as explained with reference to FIG. 8, at 910 for the current position of the implant (x/y/z-coordinate and tilt). This can be visualized at 913, for example in a cross-sectional view or perspective view as illustrated in FIG. 8. In so doing, notifications as to whether the position is correct, too low or too high can also be provided.

Finally, as likewise explained, the mechanical response of the retina (in particular mechanical deformation) to the implant can be simulated at 911, and this can be taken into account accordingly in the visualization at 914, for example by virtue of the OCT data being altered accordingly on the basis of the simulation in order to visually represent the mechanical response of the retina to the implantation.

Now, an extended method in which techniques in accordance with the present invention are also used in planning the operation is described with reference to FIGS. 10 and 11. Here, FIG. 10 elucidates the planning and FIG. 11 elucidates the assistance to the actual operation. To avoid repetition, the description of FIG. 9, already provided, is referred to in the description of FIGS. 10 and 11.

At 100, a 2D image of the retina is recorded, for example using a fundus camera or else the camera of a surgical microscope. This 2D image can be a wide-angle image with an image angle of greater than 40°, for example, which shows the entire fundus or a large part thereof. From this recording, points of interest in the retina are determined at 102, for example a point of sharpest vision, a location where the optic nerve opens into the retina, diseased regions of the retina, the course of blood vessels, and the like. In the case of a wide-angle image, the 2D image can then also serve, as it were, as a basis or map for registering various recording modalities such as OCT scans or surgical microscope images to one another, which each then only show a small section. Further information can also be included in the method of FIG. 10 or FIG. 11, e.g., data obtained from retinal angiography.

At 101, an OCT scan of the retina is made; i.e., the retina is scanned by an OCT device such as the OCT device 11 of FIG. 1 in order to consequently obtain information about the three-dimensional structure of the retina. The OCT data thus obtained are de-warped, as explained with reference to FIG. 9.

Instead of the actual operation, a virtual position (at which an avatar is then also displayed) can be entered within the scope of the planning of FIG. 10 at 103 by way of a user input, and hence it is possible, as it were, to carry out a virtual operation. To this end, use can be made of conventional input means such as a mouse or keyboard, or else of input unit means used in the field of “virtual reality”, such as gloves with motion sensors or the like. Then, the position of the implant and its tilt is determined at 104 on the basis of the user input.

At 105, an avatar of the implant is then displayed at the position just specified by the user in each case, optionally at 106 with a functional component as described. Moreover, the retina is displayed on the basis of the OCT scans at 107.

Apart from this not being a real implant but merely the display of an avatar for planning purposes, steps 105, 106, and 107 correspond to steps 95, 96, and 97, respectively, of FIG. 9.

Here, too, the same analysis and guide functions as explained with reference to FIG. 9 can be displayed, i.e., a navigation at 108, an analysis of the penetration at 109, and a simulation of the mechanical response to the implant at 1010, corresponding to steps 99, 910 and 911 of FIG. 9. Accordingly, advantageous and disadvantageous zones of the implant of the retina for implantation purposes can be visualized at 1011, information in respect of the penetration of the implant can be provided at 1012, and the simulation of the mechanical response can be visualized at 1013, corresponding to steps 912, 913, and 914 of FIG. 9. The difference once again consists in the fact that this is not related to a visualization of a currently occurring operation but related to a virtual movement of the avatar of the implant by user inputs and a display of the reaction of the retina thereto, and, consequently, a virtual operation, as it were.

The process of FIG. 10 can be implemented iteratively, i.e., on the basis of the analysis and the guide information, the user can once again alter the position at 103 and thus virtually simulate the operation procedure.

During the process of FIG. 10, the user, e.g., surgeon, can add annotations to illustrated images, visualizations, etc., at 1014, for example as a freehand drawing, symbols, labels, and the like. By way of example, this allows important points of the retina to be marked. Then, these annotations can subsequently be displayed with the visualization during the operation, as explained for the arrow 42 of FIG. 4.

The coordinates of a final position of the implant attained and points of interest of the retina thus obtained, and the annotations can then be used as output variables of the planning process of FIG. 10 and can be used as input variables during the operation to be subsequently carried out, as explained below with reference to FIG. 11.

FIG. 11 elucidates the procedure of the method during the operation if the planning of FIG. 10 was carried out previously.

At 110, like at 90 in FIG. 9, an image is recorded by means of a surgical microscope with a camera such as, e.g., the surgical microscope 12 of FIG. 1, and, at 112, like at 91 in FIG. 9, the position of the implant is found in the image. At 111, the planned position of the implant and points of interest, which are known from the planning process of FIG. 10, are moreover transferred as input data. At 1118, these points of interest are identified in the microscope image. Moreover, at 113, an OCT scan of the implant is carried out and, at 115, an OCT scan of the retina adjacent to the implant is carried out, corresponding to steps 92 and 94, respectively, of FIG. 9. These OCT data are de-warped and, at 114, the z-position of the implant is determined on the basis of the OCT scan of the implant.

Steps 116-119 in FIG. 11 correspond to steps 95-98 of FIG. 9, and reference is made to the explanations provided there. Moreover, at 1110, an outline of the implant or any other marking is displayed on the retina at the planned position. As it were, this provides the surgeon with a target for the implantation. To this end, the points of interest can serve as a reference, in respect of which the planned position is determined. Moreover, the annotations can be displayed as explained. Additionally, further data obtained in the planning phase can be used to augment the displayed visualization. Thus, the aforementioned wide-angle image can be used to display a larger region of the retina than would correspond to the viewing angle of the surgical microscope. Additionally, data emerging from the aforementioned retinal angiography can be used for augmentation purposes.

Analysis steps 1111-1113 in FIG. 11 correspond, in turn to steps 99, 910, and 911 of FIG. 9, and reference is made to the explanations provided there. To guide the operation, steps 1114-1116 correspond to steps 912-914 of FIG. 9. Additionally, at 1117 of FIG. 11, an offset can be displayed between the current position of the implant and the planned position of the implant, for example by means of arrows that point in the direction of the planned position in order thus to assist the surgeon in bringing the implant to the planned position.

Once again, reference is made to the fact that the illustrated methods only provide visual assistance during the implantation and do not relate to the surgical intervention itself.

It should likewise be emphasized, once again, that the illustrated exemplary embodiments only serve elucidation purposes and, in particular, that only some of the displayed options might be realized in some of the exemplary embodiments.

1. A method for visualizing an implantation of a retinal implant, comprising:
recording a two-dimensional (2D) image of a retina and of an implant;
carrying out an optical coherence tomography (OCT) scan of the retina and an OCT scan of the implant; and
visualizing the implant and the retina on a display on the basis of the 2D image and the OCT scan.

2. The method as claimed in claim 1, wherein the visualization of the implant comprises a display of an avatar of the implant.

3. The method as claimed in claim 2, wherein the display of the avatar comprises a display of an avatar of a structural component of the implant and an optional display of an avatar of a functional component of the implant.

4. The method as claimed in claim 2, wherein the display of the avatar comprises an optional display of the avatar in a first configuration or in a second configuration.

5. The method as claimed in claim 1, further comprising:
determining a relative position of the implant in the 2D image of the retina; and determining a scan line of the OCT scan of the retina and a scan line of the OCT scan of the implant on the basis of the determination of the relative position.

6. The method as claimed in claim 1, further comprising:
determining a distance of the implant from the retina on the basis of the OCT scan of the implant; and
displaying the distance on the display.

7. The method as claimed in claim 1, wherein the visualization of the retina comprises a visualization of a part of the retina located below the implant on the basis of a previous OCT scan.

8. The method as claimed in claim 1, wherein the visualization comprises a visualization of regions of the retina suitable for implantation purposes.

9. The method as claimed in claim 1, wherein the visualization comprises a visualization of a penetration of fastening means (80) of the implant into the retina.

10. The method as claimed in claim 9, wherein the visualization further comprises an output of an indication as to whether the penetration depth of the fastening means is correct.

11. The method as claimed in claim 1, wherein the visualization comprises a simulation of a mechanical reaction of the retina to the implant and a visualization of the simulated mechanical reaction.

12. The method as claimed in claim 1, further comprising:
prior to the implantation, carrying out a virtual operation procedure with a further visualization for establishing a planned implant position, wherein the visualization comprises a display of the planned implant position.

13. The method as claimed in claim 12, wherein the further visualization within the scope of the virtual operation is carried out on the basis of a user input for controlling the implant, a 2D image of the retina, and an OCT scan of the retina.

14. The method as claimed in claim 1, further comprising:
prior to the implantation, creating annotations, wherein the visualization comprises a display of the annotations.

15. The method as claimed in claim llany one of claim 11, further comprising:
augmenting the visualization on the basis of the data obtained prior to the implantation.

16. The method as claimed in claim 15, wherein the data obtained prior to the implantation comprise a recording of the fundus and/or data from retinal angiography.

17. An apparatus for visualizing an implantation of a retinal implant, comprising:
a surgical microscope with a camera for recording a two-dimensional (2D) image of a retina and of an implant;
an optical coherence tomography (OCT) device; and
a computing device, wherein the computing device is configured to drive the OCT device to carry out an OCT scan of the retina and an OCT scan of an implant and to drive a display to visualize the implant and the retina.

18. The apparatus as claimed in claim 17, wherein the apparatus is configured to:
record a 2D image of a retina and of an implant;
carry out an OCT scan of the retina and an OCT scan of the implant; and
visualize the implant and the retina on a display on the basis of the 2D image and the OCT scan.
